(19) United States
(12) Patent Application Publication
(10) Pub. No.: US 2007/0190655 A1
(43) Pub. Date: Aug. 16, 2007

(54) Passive type emission flux sampler and flux measuring apparatus

(75) Inventors: Yukio Yanagisawa, Tokyo (JP); Tomio Uchi, Tokyo (JP); Shaobu Cai, Tokyo (JP); Kazukiyo Kumagai, Tokyo (JP); Minoru Fuji, Ibaraki (JP)

Correspondence Address: GREENBLUM & BERNSTEIN, P.L.C., 1950 ROLAND CLARKE PLACE, RESTON, VA, 20191, US

(73) Assignees: Yukio YANAGISAWA, Tokyo (JP); FUKUWAUCHI TECHNOLOGIES INC.,, Tokyo (JP)

(21) Appl. No.: 10/597,041

(22) PCT Filed: Oct. 8, 2004
(86) PCT No.: PCT/JP04/14930

§ 371 Date: Sep. 6, 2006

(30) Foreign Application Priority Data
Jan. 9, 2004 (JP) 2004/003930

(51) Int. Cl. G01N 33/00

(52) U.S. Cl. 436/38

(57) ABSTRACT

To enable simple and accurate measurement for the flow rate (emission flux) of a chemical substance released from a site to be measured such as a floor surface, ceiling, wall surface, or furniture with no effect of external air (air in the room). [Means for Solution]

An opening 14 for taking in a chemical substance released from an inspection object (13) into the casing (12) is formed to the bottom surface (12a) of a hollow casing (12) to be bonded to the inspection object (13), a test specimen (15) that takes place color change reaction with the chemical substance under a humid circumstance is disposed to the inner surface of the casing (12) being opposed to the opening (14), and a transparent observing section (12a) is formed to the casing (12) for observing the color change of the test specimen (15) from the outside while bonding the inspection object (13) as it is.

### TECHNICAL FIELD

The present invention concerns a passive type emission flux sampler capable of simply measuring emission flux of a toxic chemical substance such as formaldehyde emission from an inspection object such as furniture and building materials into air (released amount per unit area and per unit time) with no requirement of power or electric power supply at all, and a flux measuring apparatus which measures the emission flux more correctly by using the sampler.

### BACKGROUND ART

In recent years, many cases have been reported where residents in newly built homes suffer from various physical deconditionings such as headache, sore throat, eye hurt, nasal inflammation, nausea, breathing problem, dizziness dermatitis, etc., which are called as “sick building syndrome” and bring about social problems.

The mechanism of pathogenesis of the sick building syndrome has not yet been analyzed but it may be considered to be attributable mainly to air contamination in a room due to volatilization of noxious chemicals such as formaldehyde or volatile organic compounds (VOC) contained, for example, in building materials, furniture, furnishing goods, carpets, and curtains used in the residences.

By the way, in a case where residents in newly built homes suffer from sick building syndrome, or where room contamination at high concentration, not restricted to the newly built homes, is found, if it can be determined which building material or furniture releases the causal substance, causes for the sick building syndrome can be eliminated by replacing the relevant building material or the furniture.

However, the method of measuring the released amount of the volatile organic chemical substance according to JIS at present is a desiccator method of measuring a test specimen of the building material by putting it into a small-sized desiccator and, while it is an urgent need for preparing a draft for a small-sized chamber method of using a small-sized chamber of 20 to 1000 liter capable of accommodating and measuring building materials, or a large-sized chamber method of using a large-sized chamber capable of accommodating and measuring furniture or building materials for the feature, none of them can measure the emission flux from the building materials assembled to the building.

Further, while apparatus for measuring the concentration of noxious chemical substances contained in air in the room are present, since the measuring apparatus can not measure the emission flux of the noxious chemical substance, the source of release can not be identified.

Accordingly, it has been proposed recently measuring apparatus for the amount of chemical substances released from any place such as walls, ceilings, and floors by attaching an attachment to the concentration measuring apparatus.
[Patent Document 1] JP-A No. 2002-162322.

FIG. 5 shows such an existent measuring apparatus 41, in which the bottom of an attachment 42 formed in a box-like configuration is formed as an opening 43, clean air introduction ports 45 provided with filters, etc. are formed on the lateral surfaces 44, and an air exit port 46 is formed at the upper surface, and a concentration measuring apparatus 47 for automatically sucking air and measuring the concentration of chemical substances contained in air is connected with the air exit port 46.

Then, when air is sucked by the concentration measuring apparatus 47 in a state of engaging the opening 43 of the attachment 42 to the site of the inspection object such as a wall surface, ceiling surface or floor surface, noxious chemical substances released from the wall surface, etc. are measured by the concentration measuring apparatus 47.

However, since the box 41 is as large as: length×width×height=20 cm×20 cm×30 cm in the size in relation with the air suction amount of the concentration measuring apparatus 47, it is inconvenient to carry about and expensive as well, so that measurement is conducted usually by a set of the measuring apparatus 41.

Accordingly, it requires a long time survey for identifying the source of release for which indoor measurement at a plurality of points is necessary in the building.

For example, in a case where the source of release of chemical substances in one room, is intended to identify, measurement has to be conducted for at least several sites such as on the wall, ceiling, floor, at the room doors, or in the closet. In this case, since measurement has to be conducted successively by using a set of the measuring apparatus 41 and it needs at least about 30 min for the measurement of one site, in a case where all the rooms are intended to be measured thoroughly in one newly built building, this results in a problem of consuming much time and labor.

Further, since the opening of the attachment 41 is as large as: 20 cm×20 cm, measurement can not be conducted unless the place has a plane at least corresponding to the size and since the height is as large as 30 cm, it involves a problem that the measurement is impossible for the narrowed portion with a structural view point of the building.

In addition, since the attachment 42 is lined with stainless steel at the inside and is heavy, it is extremely difficult to be fixed on the ceiling or wall surface and only the floor surface can be actually measured. In addition, since it has a structure of taking in external air (indoor air) from the clean air introduction port 45 formed on the lateral surface 44, in a case where the indoor air has already been contaminated with chemical substances they can not be removed by the filter but intrude into the attachment, this also results in a problem that the reliability for the result of measurement is low.

Further, the air flowing state on the surface of the site of the inspection-object i.e. wall surface, ceiling surface, floor surface etc., to which the opening 43 is contacted, is different from the usual state, since the measuring method is an active type to suck air automatically from the exit port 46.

That is, since the flow speed of air on the surface of the site for the inspection object is increased more compared with that in the usual state of use, the diffusion mechanism of the noxious chemical substance changes from a rate limiting step of the mass transfer in a gas phase boundary layer near the surface of the inspection object to that inside the inspection object

Accordingly, the method of a passive type which can maintain the gas flow in the usual state during measurement is recently recommended, since the measurement result by this method such as an active type is different from the emission flux in the usual state of use.

### DISCLOSURE OF THE INVENTION

### Subject to be Solved by the Invention

Then, the present invention has a technical subject of providing a passive type emission flux sampler capable of simply and accurately measuring the released flow rate (emission flux) of a chemical substance released from a site to be measured including a floor surface, as well as a ceiling, a wall surface, or even a narrow place, not undergoing the effect of external air (indoor air) or without disturbing the flowing state on the surface of the measuring site.

### Means for Solving the Subject

For solving the subject, a passive type emission flux sampler according to the present invention is adapted to measure a emission flux of a certain chemical substance released from an inspection object into air, in which an opening for taking in a chemical substance released from the inspection object into the casing is formed to a bottom surface of a hollow casing to be bonded to an inspection object in a state of bonding the bottom surface to the inspection object, a test specimen that takes place color change reaction with the chemical substance under a humid circumstance is disposed to the inner surface of the casing of an opposite side of the opening, and the casing has a gas barrier property.

Further, the emission flux measuring apparatus according to the present invention is a emission flux measuring apparatus of a passive type flux sampler using a test specimen that takes place color change reaction with a specified chemical substance under humid circumstance, in which

the flux sampler is formed with an opening at the bottom surface of a hollow casing to be bonded to an inspection object for taking in a chemical substance released from an inspection object into the casing, a test specimen that takes place color change reaction with the chemical substance in a humid circumstance is disposed to the inner surface of the casing being opposed to the opening, and the casing is formed with a transparent observing section for observing the color change of the test specimen from the outside in a state being bonded to the inspection object,

a light shielding chamber formed with a setting stage for positioning the flux sampler reacted for a predetermined time is provided with an light source for irradiating a measuring light to the observing section of the flux sampler, and an optical sensor for detecting the intensity of a reflection light from the observing section of flux sampler, and

a calculation processing device for calculating the emission flux based on the intensity of the reflection light detected by the optical sensor is provided.

### EFFECT OF THE INVENTION

According to the passive type emission flux sampler of the present invention, when the test specimen is wetted by dropping water and then the bottom surface of the casing is bonded and fixed to the inspection site of any inspection object such as wall surface, ceiling surface, or floor surface, since a noxious substance such as formaldehyde or volatile organic compound (VOC), when it is contained in the inspection object, intrudes from the opening into the casing and reaches the test specimen, the test specimen changes the color in accordance with the emission flux (released flow rate) of the noxious substance

Accordingly, by comparing the color of the test specimen with a color chart formed previously in accordance with the emission flux, the emission flux of the noxious substance from the inspection site can be measured, and the total amount of release discharged from the entire building materials can also be calculated based on the ratio between the opening area of the opening and the area of the entire building material.

In this case, since the emission flux is measured by utilizing the color change reaction of the test specimen and observing the color change thereof, any power or electric supply is not necessary at all.

In this case, since the hollow casing has gas garrier property, and the bottom surface with opening is contacted with the inspection object, and the inside of the casing is shielded from the external air, even when the indoor air is contaminated with the organic substance, only the emission flux of the noxious substance released from the inspection object can be detected accurately not undergoing the effect thereof.

Further, since not an active method of transporting the object noxious substance to the test specimen by suction of air using power but by a passive method of transporting the noxious substance as far as the test specimen by molecule diffusion of the object noxious substance caused in the spontaneous state is utilized, the emission flux can be measured accurately in the usual state of use without disturbing the flowing state on the surface by measurement.

While some of the noxious substances may permeate through the casing at a certain permeation rate, the permeation rate of the noxious substance can be kept lower by forming a DLC film on one surface or both surfaces of the casing for such substance.

Further, while the sampler may be of any size, in a case of using a square sample of about 5 mm to 1 cm in for length and the width, it may suffice that the outer profile size of the hollow casing is utmost about: length×width×thickness=2 cm×2 cm×3 cm and it can be simply bonded and fixed even to any narrow portion by using a double-sided pressure sensitive adhesive tape or the like.

Further, since the individual sampler is extremely simple in the structure and the manufacturing cost therefor is inexpensive as well, emission fluxes can be measured simultaneously by bonding and fixing a plurality of samplers to respective measuring sites.

It is not restricted to a case of measuring the emission flux by comparing the color of the test specimen with the color chart after lapse of a predetermined time but also measurement can be made more accurately by optically measuring the color of the test specimen and conducting calculation based thereon.

In this case, when the flux sampler put to reaction for a predetermined time is set to the setting stage formed in the light shielding chamber of the measuring apparatus, the measuring light irradiated from the light source is irradiated to the observing section, and the intensity of the reflection light is detected by the optical sensor.

The intensity of the reflection light corresponds to the color of the test specimen and the color of the test specimen corresponds to the emission flux.

Accordingly, by previously determining a relation between the emission flux and the intensity of the reflection light, the emission flux can be calculated accurately based on the intensity of the detected reflection light.

### BEST MODE FOR PRACTICING THE INVENTION

The present invention has attained the subject of enabling simple and accurate measurement for the flow rate of a chemical substance released from a site to be measured without undergoing the effect of external air (indoor air), by using a sampler of an extremely simple constitution without using an electric measuring apparatus.

### EMBODIMENT 1

The present invention is to be described specifically by way of a preferred embodiment for practicing the invention with reference to the drawings.

FIG. 1 is a cross sectional view showing an example of a passive type emission flux sampler according to the invention.

A passive type emission flux sampler 1 of this embodiment is adapted to measure emission flux (released flow rate) in a casing where formaldehyde (chemical substance) contained in an inspection object 3 such as a building material is released in air, in which an opening 4 for taking in formaldehyde released from an inspection-object 3 into an casing 2 is formed to a bottom surface 2a of the hollow casing 2 with gas garner property, and a test specimen 5 taking place color change reaction with formaldehyde under a humid circumstance is disposed being opposed to the opening 4 at the inner surface of the casing 2.

The opposite surface to the bottom surface 2a of the casing is an observing section 2b for observing the color change of the test specimen 5 from the outside.

In the test specimen 5, INT (p-iodo-nitrotetrazolium violet) as a chromophoric agent and two types of enzymes dehydrogenase and diaphorase as a reaction catalyst are carried on a paper substrate sheet, for example, of about 1 cm×1 cm size.

Thus, when formaldehyde is in contact with the test specimen 5 wetted with water, hydrogen of formaldehyde is dissociated by the dehydrogenase and decomposed into formic acid and NADH (nicotinamide adenine dinucleotide) and NADH and INT are reacted by diaphorase to decrease INT and develop a color.

An air permeable spacer 6 of a predetermined thickness is provided between the opening 4 and the test specimen 5 for ensuring a predetermined distance (for example, 1 mm) between the surface of the inspection object 3 and the test specimen 5, which is formed of a porous material capable of allowing formaldehyde released from the inspection object 3 to reach the test specimen 5 and this is formed, for example, of metal or plastic perforated with a number of vent holes.

Further, a pressure sensitive adhesive layer 7 such as a double-sided tape is formed on the bottom surface 2a of the casing 2, and the thickness of the air permeable spacer 6 is selected such that the test specimen 5 is urged to the air permeable spacer 6 when it is bonded and fixed to the surface of the inspection object 3, and the air permeable spacer 6 is urged to the surface of the inspection object 3 so as not to cause a gap between the casing 2 and the inspection object 3.

Then, the casing 2 is formed to such an entire size, for example, of about: length×width×thickness=2 cm×2 cm×3 mm and the recess 5 is formed to such a size of about: length×width×depth=1 cm×1 cm×1.5 to 2 mm.

In a case of using a plastic casing 2 of such a thickness, since formaldehyde permeates the plastic, a gas barrier film 8 such as a transparent DLC film (diamond like carbon film) or vapor deposited silica film is vapor deposited at least on one of the outer surface or the inner surface of the casing 2 in order to enhance the gas barrier property against formaldehyde. A DLC film is formed in this embodiment.

Since the DLC film has an extremely high gas barrier property to formaldehyde, formaldehyde contained in air in the room does not permeate the casing 2 and discolor the test specimen 5, but only the emission flux of formaldehyde released from the inspection object 3 can be measured exactly.

For the hollow casing 2, any material can be used such as glass not being restricted to the plastic material and, in a case of using glass, since the gas barrier property is high by nature, it is not necessary to form a gas barrier film.

A constitutional example of the invention is as has been described above and the operation thereof is to be described.

At first, water is dropped on the side of the air permeable spacer 6 of the passive type emission flux sampler 1 preserved air tightly to wet the test specimen 5.

Then, the casing 2 is bonded and fixed by the pressure sensitive adhesive tape or the like with the side of the air permeable spacer 6 being directed to the inspection object 3 such as a wall surface, floor surface, ceiling surface, or the surface of furniture.

Then, when it is left as it is for a predetermined time, formaldehyde released from the inspection 3 permeates the air permeable spacer 6 and reaches by way of molecule diffusion to the test specimen 5 spaced apart by a predetermined distance.

Accordingly, when the amount of the emission flux is large, the chromogenic reaction is promoted and the test specimen 5 exhibits deep red and blue color and in a case where the amount of the emission flux is small, the test specimen 5 exhibits pale red purple color by the chromogenic reaction.

Since the color of the test specimen 5 changes as described above, by comparison with a color chart previously prepared in accordance with the emission flux for the color after lapse of a predetermined time, emission flux of a noxious substance from the inspection site for the inspection object 3 can be measured.

Further, for an identical material, since it can be estimated that the amount of the emission flux from other portions is also identical, a total amount of release can be calculated based on the ratio between the area of the air permeable spacer 6 and the surface area of the inspection object 3.

In this case, since the emission flux is measured by observing the color change utilizing the color change reaction of the test specimen 5, an inhalation of air is not necessary, and neither power nor electric power supply is not necessary at all upon measurement.

In this case, since the test specimen 5 and the air permeable spacer 6 are covered by the casing 2 in a lamination state, and engages the inspection object in a state sealed from external air, even when air in the room is contaminated with noxious substances, only the noxious substances released from the inspection object can be detected accurately without suffering from the effect thereof.

Further, since a predetermined distance of space can be ensured between the test specimen 5 and the inspection object 3 by the air permeable spacer 6, measurement can be conducted always under the same condition.

Further, since the noxious substance as an object is transported by molecule diffusion as far as the test specimen, emission flux can be measured accurately in a usual state of use without disturbing the flowing state on the surface of the measuring site by measurement.

Since the casing 2 has the DLC film 8 formed on one or both of the outer surface and the inner surface thereof and has a high gas barrier property against formaldehyde, formaldehyde contained in air in the room does not permeate the casing 2 and discolor the test specimen 5 but the emission flux of formaldehyde released from the inspection object 3 can be measured accurately.

Further, since the sampler 1 can be formed in an extremely small size as described above, it can be simply bonded and fixed to any narrow place by using, for example, a pressure sensitive adhesive tape.

Further, since the individual sampler 1 has an extremely simple structure and the production cost thereof is inexpensive, emission fluxes at a number of measuring sites can be measured simultaneously by bonding and fixing a plurality of samplers 1 to respective measuring places.

### EMBODIMENT 2

FIG. 2 is an explanatory view showing another embodiment of a passing type emission flux sampler according to the invention.

A passive type emission flux sampler 11 of this embodiment includes a hollow-casing 12 with gas garrier property formed into a hollow disk-like shape, the bottom surface 12a is formed with an opening 14 for taking a chemical substance released from the inspection object 13 into the casing 12 in a state of bonding the bottom surface 12a to the inspection object 13, and a test specimen 15 exhibiting color change by reaction with a chemical substance under a humid circumstance is bonded to the inner surface of the casing 12 of an opposite side of the opening 14.

Thus, a distance from the surface of the inspection object 13 to the test specimen 15 can be kept constant in a state of bonding the flux sampler 11 to the inspection object 13.

Further, the hollow casing 12 is entirely formed transparent such that the color change of the test specimen 15 can be observed from the outside in a state of bonding to the inspection object 13 as it is, and the side opposite to the bottom surface 12a constitutes an observing section 12b for observing the test specimen 15 from the rear face, and a flange 12c is formed to the outer peripheral edge such that bonding and detachment can be conducted easily.

In the casing 12, an annular water retaining paper (water retaining material) 16 is disposed so as to surround a flow channel from the opening 14 to the test specimen 15, which sucks a water droplet upon dripping the water droplet from the opening 14 into the casing 12 during measurement to keep the test specimen 15 in a humid circumstance.

Further, an annular rib 17 extends from the end edge of the opening 14 to the inside of the casing 12, by which the water droplet dripped from the opening 14 is guided with no stagnation by the surface tension of the water droplet to the water retaining paper 16 and guides the chemical substance released from the inspection object 13 straight to the test specimen 15 disposed being opposed to the opening 14 and causes the color change reaction more accurately in accordance with the released amount thereof.

In this embodiment, the hollow casing 12 is made of a plastic material of about 0.5 mm thickness to about: diameter ×thickness=2 cm×3 mm and a diameter of opening 14 of about 5 mm.

In a case of using the plastic casing 12 of such a thickness, since formaldehyde permeates the plastic, a gas barrier film 8 such as a transparent DLC film (diamond like carbon film) or vapor deposited silica film is vapor deposited at least on one of the outer surface or the inner surface of the casing 12 in order to enhance the gas barrier property against formaldehyde. A DLC film is formed in this embodiment.

Since the DLC film has an extremely high gas barrier property to formaldehyde, formaldehyde contained in air in the room does not permeate the casing 12 and discolor the test specimen 15 but only the emission flux of formaldehyde released from the inspection object 13 can be measured accurately.

For the hollow casing 12, any material can be used such as glass or the like not being restricted to the plastic material and, in a case of using glass and, since the gas barrier property is high by nature, it is not necessary to form a gas barrier film.

Then, an annular adhesive layer 19 is formed at the periphery of the opening 14 at the bottom surface 12a of the hollow casing 12, and a circular aluminum sheet 20 is bonded to the adhesive layer 19 to air tightly seal the opening 14 so that moisture does not intrude into the casing 12 in a preserved state.

In a case of measurement by using the flux sampler 11, the aluminum sheet 20 is peeled, a water droplet is dripped from the opening 14 into the casing 12 to moisten the test specimen 15, and a water retaining paper 16 is wetted so as to maintain the test specimen 15 in a humid circumstance during measurement.

In this case, since the annular rib 17 is formed to the opening 14, the water droplet flows smoothly into the casing 2 without staying at the end edge of the opening 14 by the surface tension of the water droplet.

Then, the bottom surface 12a of the casing 12 is bonded to any inspection object 13 such as a wall surface, floor surface, ceiling surface, or furniture.

In this case, even when it is bonded with the opening 14 being downward, since the water droplet in the casing 12 is dammed by the annular rib 17 formed to the opening 14, it does not flow out from the opening 14.

In this state, the chemical substance released from the inspection object 13 passes through the opening 14 and is taken into the casing 12, guided along the flow channel formed with the annular rib 17 and reaches the test specimen 15 disposed in front thereof.

Then, after lapse of a predetermined time (30 min to 2 hours), the test specimen 15 turns to deep red color in a place where the amount of the emission flux is large and turns to pale red color in a place where it is small, and scarcely changes in a place where it is nearly equal to 0.

Accordingly, the emission flux can be measured in accordance with the color of the test specimen 15 in the same manner as described above.

FIG. 4 is a measuring apparatus of emission flux for calculating the emission flux according to the invention.

A measuring apparatus 21 of this embodiment is adapted to measure the emission flux by using a flux sampler 11 described previously, in which a light shielding chamber 23 is formed to the inside of a light shielding cap 22 for optically measuring the color change of a test specimen 15 and includes a calculation processing device 24 for calculating the emission flux based on the detected color change and a liquid crystal display 25 for displaying the value thereof.

In the light shielding chamber 23, are disposed a setting stage 26 for positioning the flux sampler 11, a light source 27 for irradiating a measuring light to the observing section 12b of the flux sampler 11, and an optical sensor 28 for detecting the intensity of reflection light from the observing section 12b of the flux sampler 11.

When the flux sampler 11 is set to the setting stage 26 with the observing section 12b being downwarded, a measuring light is irradiated from the light source 27 disposed below the setting stage 16 to the position for the test specimen 15.

Since the test specimen 15 reacts with formaldehyde to discolor to red to red and purple color, the light source 27 uses an LED that outputs, as a measuring light, a green light in a complementary color relation therewith, and the center wavelength of the measuring light is selected to 555 nm in this embodiment.

A photodiode having a peak sensitivity at a wavelength of 500 to 600 nm is used as the optical sensor. In a case where the amount of emission flux of formaldehyde is large, since the test specimen 15 changes to a deep color to absorb the measuring light, the intensity of the reflection light detected by the optical sensor 28 is lowered whereas, in a case where the amount of the emission flux is small, since the test specimen 15 is less discolored and absorbs less measuring light, the intensity of the reflection light increases relatively.

The calculation processing apparatus 24 calculates the degree of absorption along with discoloration based on the intensity of the reflection light to calculate the amount of release based on the degree of light absorption.

At first, the light absorption degree P is calculated according to the following equation;
P=[1−V₁V₀]×100%
V₀ : intensity of reflection light of the test specimen 15 before reaction, or standard white light.

V₁ : intensity of reflection light for the test specimen 15 after reaction.

Then, a relation between the amount of released Fn and the light absorption degree Pn is stored based on the light absorption degree Pn of the sampler 11 measured by a known standard released amount Fn in a light absorption degree-released amount translation table 29, and the released amount Fn is determined with reference to the light absorption degree-released amount translation table 29 based on the light absorption degree P calculated for the flux sampler 11 after the reaction.

The light absorption degree-released amount translation table 29 may be in a state where it is represented by a function: Fn=f(Pn) or in a state of tabulating where the translated values and storing them.

With such a constitution, since the released amount P can be outputted as a numerical value, the released amount can be calculated accurately for a subtle color change of the test specimen 15 even in a case where comparison with the color chart is difficult.

While description has been made to a case where a transparent observing section 12b is formed to the casing 12, the invention is not restricted only thereto but it may be not transparent. In this case, when it is measured optically by using the measuring apparatus 21, a measuring light may be irradiated to the test specimen 12 on the side of the opening 14.

### INDUSTRIAL APPLICABILITY

As has been described above, the present invention is applicable to the measurement of emission flux of formaldehyde, as well as the invention is not restricted only thereto but applicable to an application use of measuring emission flux of other chemical substances such as volatile organic compounds (VOC) by optionally selecting a reagent to be impregnated into the test specimen.

### BRIEF DESCRIPTION OF THE DRAWINGS

FIG. 1 is an explanatory view of a passive type emission flux sampler according to the invention.

FIG. 2 is an explanatory view showing another embodiment of the invention.

FIG. 3 is an exploded constitutional view thereof.

FIG. 4 is an explanatory view of a measuring apparatus for emission flux according to the invention.

FIG. 5 is an explanatory view showing an existent apparatus,

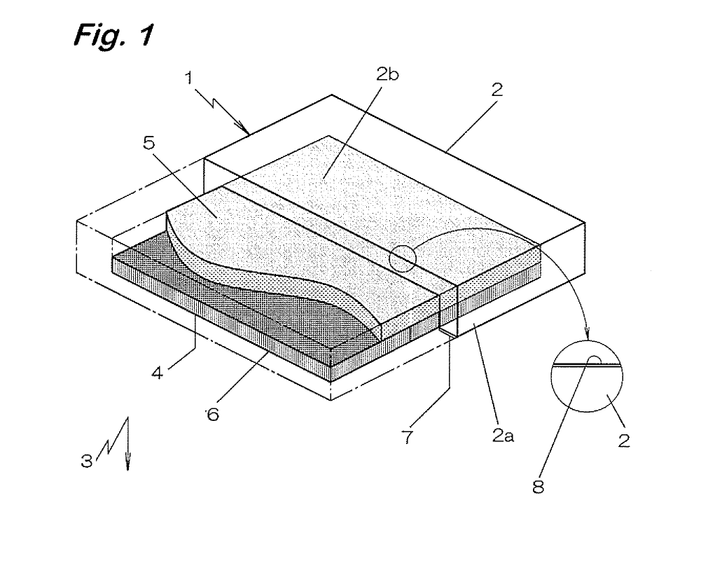

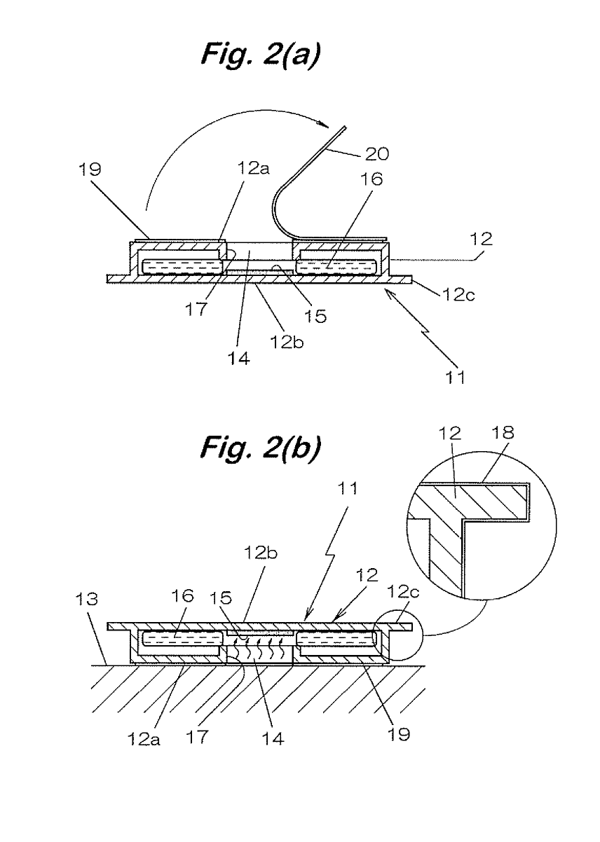

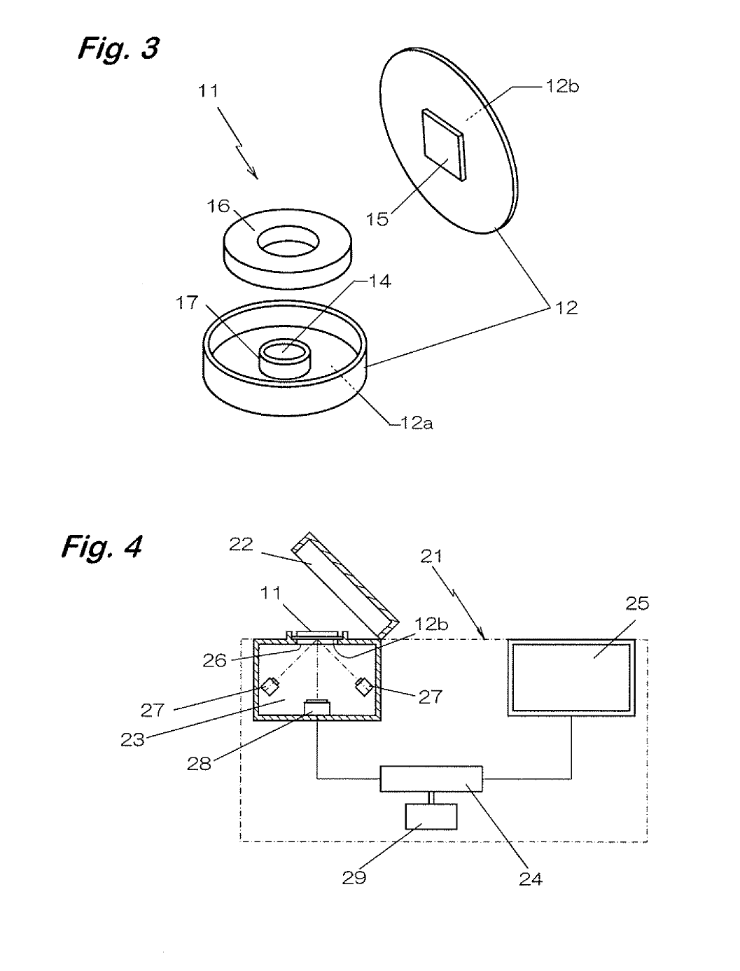

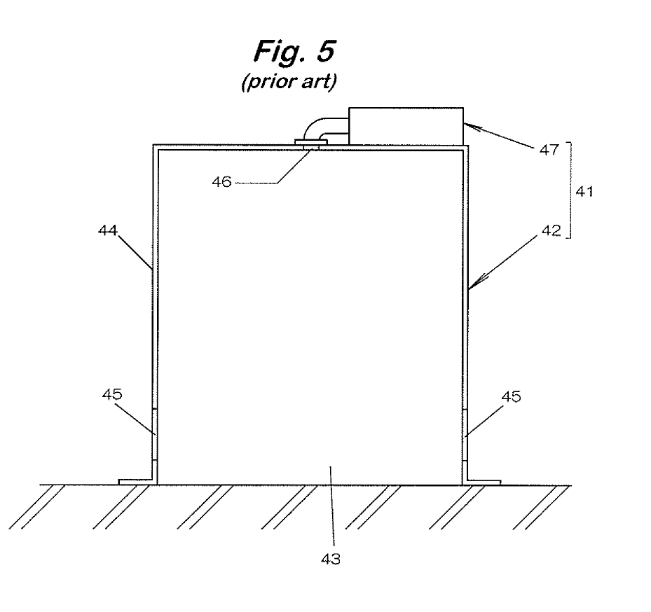

### DESCRIPTION FOR REFERENCES

1, 11 passive type emission flux sampler
2, 12 casing
2a, 12a bottom surface
2b, 12b observing section
3, 13 inspection object
4, 14 opening
5, 15 test specimen
6 air permeable spacer
7, 19 pressure sensitive adhesive layer
8, 18 DLC film
16 water retaining material
17 annular rib
21 measuring apparatus for emission flux
22 light shielding cap
23 light shielding chamber
24 operation processing device
25 liquid crystal display
26 setting stage
27 light source
28 optical sensor
29 translation table for light absorption degree-released amount

1-16. (canceled)

17. A passive type emission flux sampler for measuring an emission flux of a specified chemical sample released from an inspection object, comprising:
a hollow casing comprising an inner surface and a bottom surface opposite the inner surface in which an opening is formed for taking in a chemical substance released from the inspection object; and
an adhesive layer formed at a periphery of the opening and configured to bond the bottom surface to a surface of the inspection object,
wherein the inner surface is configured to receive a test specimen that changes color in a reaction with the chemical substance.

18. A passive type emission flux sampler according to claim 17,
wherein the inner surface comprises a transparent observing section for observing the color change of the test specimen from the outside.

19. A passive type emission flux sampler according to claim 17,
wherein the casing has a gas barrier property.

20. A passive type emission flux sampler according to claim 17,
wherein a gas barrier film is formed to at least one of the inner surface and an outer surface of the casing to provide the casing with a gas barrier property.

21. A passive type emission flux sampler according to claim 17, further comprising:
a water retaining material disposed in the casing for maintaining the test specimen in a humid condition.

22. A passive type emission flux sampler according to claim 17, further comprising:
an annular rib extending from an end edge of the opening to inside the casing.

23. A passive type emission flux sampler according to claim 17,
wherein an air permeable spacer of a predetermined thickness is configured to ensure a predetermined distance between the opening and the test specimen.

24. A measuring apparatus for measuring emission flux of a passive type flux sampler, comprising:
a hollow casing comprising an inner surface having a transparent observing section and a bottom surface opposite the inner surface in which an opening is formed for taking in a chemical substance released from an inspection object, and an adhesive layer formed at a periphery of the opening and configured to bond the bottom surface to a surface of the inspection object, wherein the inner surface is configured to receive a test specimen that changes color in a reaction with the chemical substance,
a light shielding chamber formed with a setting stage for positioning the flux sampler for a predetermined time;
a light source provided to the light shielding chamber for irradiating a measuring light to the test specimen through at least one of the opening and the observing section,
an optical sensor configured to detect the intensity of a reflection light from the test specimen, and
a calculation processor configured to calculate the emission flux based on the intensity of reflection light detected by the optical sensor.

25. A passive type emission flux sampler according to claim 19,
wherein a gas barrier film is formed to at least one of the inner surface and an outer surface of the casing to provide the casing with a gas barrier property.

26. A passive type emission flux sampler according to claim 18, further comprising:
a water retaining material disposed in the casing for maintaining the test specimen in a humid condition.

27. A passive type emission flux sampler according to claim 19, further comprising:
a water retaining material disposed in the casing for maintaining the test specimen in a humid condition.

28. A passive type emission flux sampler according to claim 18, further comprising:
an annular rib extending from an end edge of the opening to inside the casing.

29. A passive type emission flux sampler according to claim 19, further comprising:
an annular rib extending from an end edge of the opening to inside the casing.

30. A passive type emission flux sampler according to claim 18,
wherein an air permeable spacer of a predetermined thickness is configured to ensure a predetermined distance between the opening and the test specimen.

31. A passive type emission flux sampler according to claim 19,
wherein an air permeable spacer of a predetermined thickness is configured to ensure a predetermined distance between the opening and the test specimen.

32. A passive type emission flux sampler according to claim 17,
wherein a flange is formed to an outer peripheral edge of the casing.

33. A passive type emission flux sampler according to claim 17,
wherein the adhesive layer is configured to bond to an aluminum sheet to seal the opening.
